(19) United States
(12) Patent Application Publication
(10) Pub. No.: US 2016/0256250 A1
(43) Pub. Date: Sep. 8, 2016

(54) Method for the Prevention of Incontinence During a Prostatectomy by Means of a Textile Implant

(71) Applicant: ASPIDE MEDICAL, La Talaudiere (FR)

(72) Inventor: Andrea Cestari, Milano (IT)

(21) Appl. No.: 14/640,892

(22) Filed: Mar. 6, 2015

(51) Int. Cl. A61F 2/00; A61M 25/10

(52) U.S. Cl. CPC A61F 2/0045; A61M 25/10; A61B 2017/00274

(57) ABSTRACT

The method for the prevention of incontinence during a prostatectomy, by means of a textile implant comprises a support portion of the urethra fitted with at least 2 arms, a method according to which: after performing the prostatectomy, the implant is positioned, at the time of reconstruction of the urinary contiguity, without tension in the surgical area; the rectovesical fascia is reconstructed to create an anatomical barrier/separation between the implant and the ureterovesical anastomosis; anastomosis is performed and an indwelling catheter is inserted; the arms are tensioned and fixed to the Cooper ligament and/or the psoas muscles; a balloon catheter is inflated and the surgical procedure is conventionally completed.

### TECHNICAL FIELD

The disclosure relates to the technical sector of implants for the treatment or the prevention of urinary incontinence in men. Such incontinence may result, for instance, from a prostatectomy performed for the treatment of prostate cancer.

### BACKGROUND

In a perfectly known manner for this type of urinary incontinence, it is common to use textile implants, having a central urethra support portion, symmetrically extending in the form of at least two transobturator or retro/pubic arms arranged in the same plane and in opposition.

In the patent FR 2 959 116, the central support portion also has pre-pubic arms, arranged according to a “V” orientation, with respect to one another, and with respect to the subjacent transobturator arms.

This type of implant is used during a surgical operation for the treatment of incontinence, following a prior operation for a prostatectomy, corresponding as a consequence, to two dissociated surgical operations.

Incontinence following a prostatectomy is frequent and as its natural recovery can be long and socially disabling, the aim sought, according to the disclosed embodiments, is to speed up this recovery from incontinence, after a radical prostatectomy, using an implant, of the type of those designed for the post-chirurgical treatment of male incontinence (support of bulbar urethra with perineal insertion).

The problem posed is therefore to treat on the one hand the urethra, at the external urinary sphincter, and on the other hand the bladder neck, in order to restore a normal anatomical shape, after a radical prostatectomy (i.e. by accounting for the absence of the prostate), or to suitably support the orthotopic neobladder and the external urinary sphincter, following a cystoprostatectomy.

### SUMMARY

The aim sought, according to the disclosed embodiments, is therefore to prevent post-prostatectomy incontinence during the operation itself and no longer treat it after a prostatectomy.

To overcome such a problem, and achieve this objective, an incontinence prevention method during a prostatectomy was developed, by means of a textile implant comprising a urethra support portion fitted with at least two tapes or arms, a method according to which:
after performing the prostatectomy, the implant is positioned, at the time of reconstruction of the urinary contiguity (ureterovesical anastomosis) without tension in the surgical area,
the rectovesical fascia is reconstructed to create an anatomical barrier/separation between the implant and the ureterovesical anastomosis,
anastomosis is performed and an indwelling catheter is inserted,
the tapes/arms of the implant are tensioned and fixed to the Cooper ligament just lateral to the pubic simphysis,
a balloon catheter is inflated and the surgical procedure is conventionally completed

This surgical treatment method may be implemented by different types of implants.

When the implant has from the central support portion, in a symmetrical manner, two anterior tapes or anterior arms and two posterior tapes, the anterior tapes are fixed to the Cooper ligament just lateral to the pubic simphysis and the posterior tapes to the psoas muscle/Cooper ligament (depending on patient body shape and anatomy) in the area between the obturator nerve and the external iliac vessels.

When the implant has, from the central support portion, in a symmetrical manner, two anterior tapes and four posterior tapes, the two anterior tapes are fixed to the Cooper ligament just lateral to the pubic simphysis and the four posterior tapes to the psoas muscle/Cooper ligament (depending on patient body shape and anatomy) in an area between the obturator nerve and the external iliac vessels.

The disclosed embodiments also relate to an implant for the implementation of the method as claimed.

Thus, in one embodiment, the implant symmetrically has, from a central support portion, two posterior arms arranged in the same plane, and in alignment, and two anterior arms, in a “V” configuration, with respect to one another, and with respect to the posterior arms.

In another embodiment, the implant claimed symmetrically has, from a central support portion at least 2 arms and ideally six arms arranged in the same plane and in alignment.

### BRIEF DESCRIPTION OF THE FIGURES

Various embodiments are set out below in more detail with the help of the appended figure drawings in which:

FIG. 1 is a view of one embodiment of an implant with four arms;

FIG. 2 is a view of one embodiment of an implant with six arms;

FIGS. 3 to 6 show an implant positioning method, in compliance with that illustrated in FIG. 2, and the positioning passageway of the said implant with respect to the pelvis;

FIG. 7 is a schematic view of an implant, according to the positioning method, according to the disclosure; and

FIGS. 8 and 9 show two alternative embodiments of the implant, of the type of that illustrated in FIG. 2.

### DETAILED DESCRIPTION

The drawing figures illustrate a urethra support implant (1) for the treatment of male urinary incontinence, in the form of a tape, knitted, woven or otherwise processed, made of a polypropylene or similar material, biocompatible, to be positioned under the urethra of the patient, as indicated.

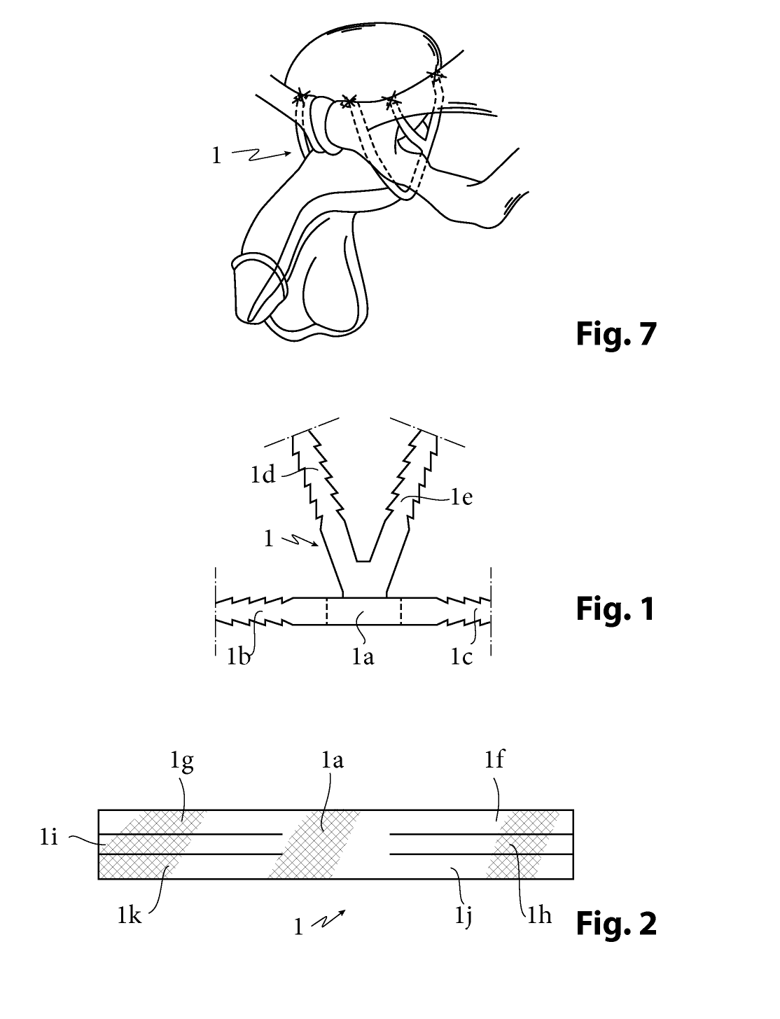

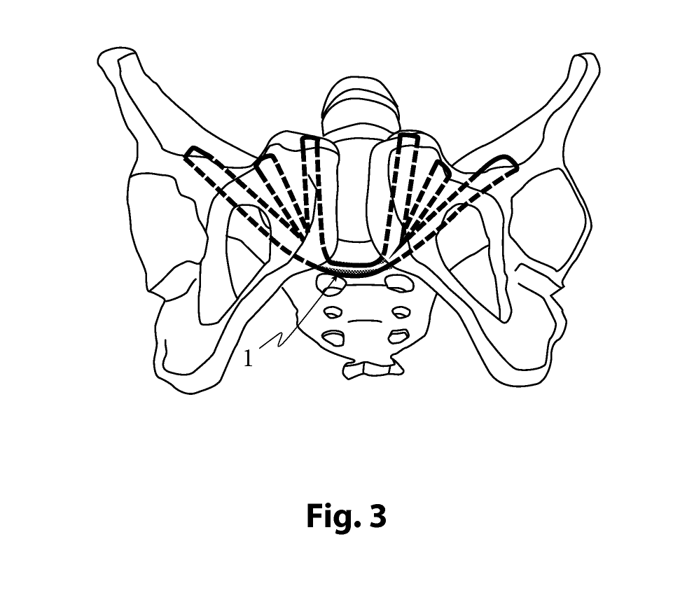

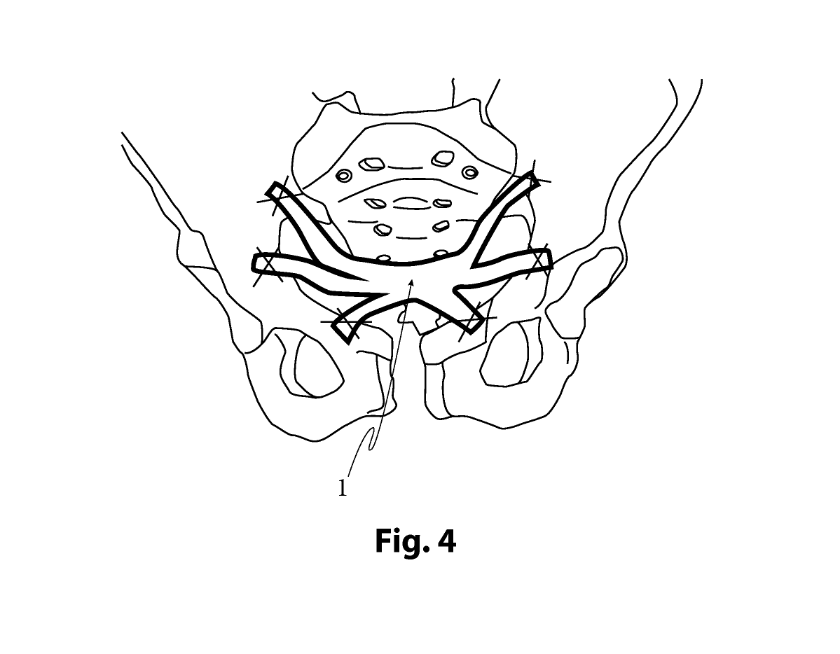

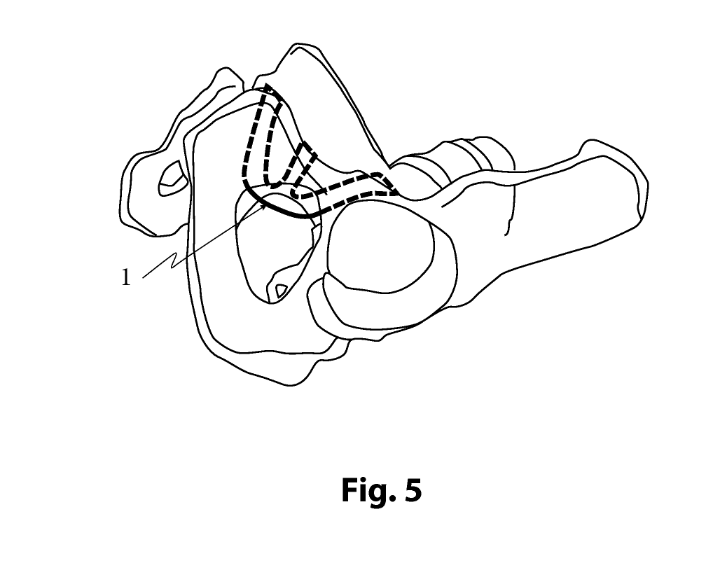

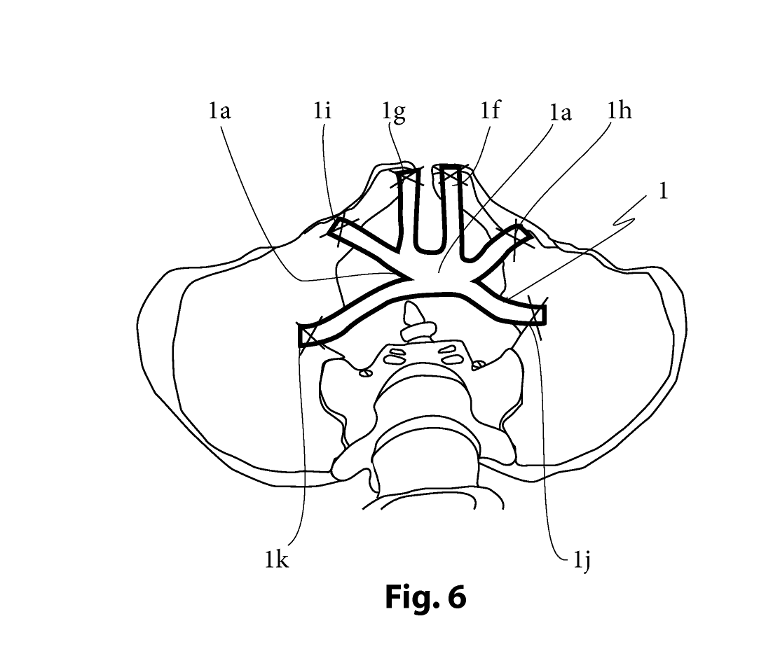

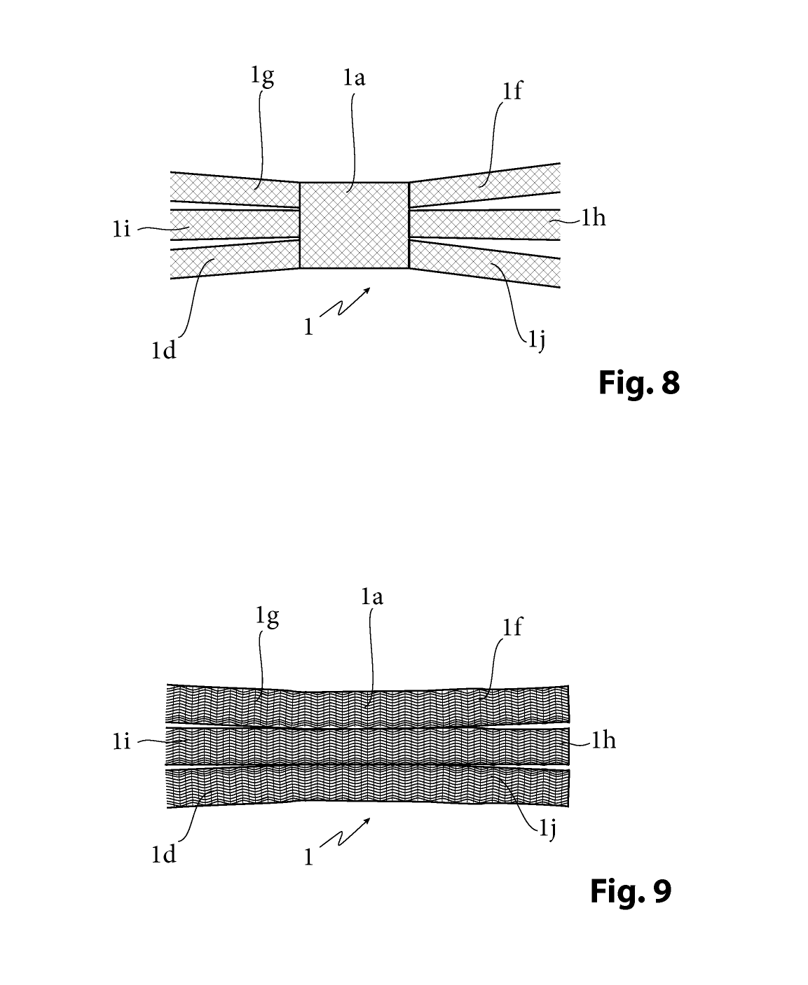

In the embodiment illustrated in FIG. 1, the implant (1) comprises a central portion (1a) to support the urethra. This central support portion (1a) symmetrically has two posterior arms (1b) and (1c) arranged in the same plane and in alignment. The central support portion (1a) is extended from the top by a projecting section, from which are arranged two anterior arms (1d) and (1e) positioned according to a “V” configuration, with respect to one another, and with respect to the posterior arms (1b) and (1c). This type of implant is made apparent from the teaching of the patent FR 2 959 116.

In the embodiment illustrated in FIG. 2, the implant (1) symmetrically has, from the central support portion (1a), six arms (1f) (1g) (1h) (1i) (1j) (1k), arranged in the same plane and in alignment. In an alternative embodiment, the implant (1) is constituted of 3 tapes (for instance knitted) assembled side by side in their centre by any means such as a seam, over a sufficient length (for example 4 cm) to constitute the central part (1a) of the implant (FIG. 9).

Similarly, the implant (1) may have coloured marks to identify the central part (1a) during the operation by means for example of two marks (e.g. sewn) at the two ends of the said central part (to avoid a potentially irritating overthickness under the urethra) (FIG. 10).

It is furthermore pointed out that the tapes are atraumatic, hardly extensible and adapted to contact with the urethra (e.g. of the female stress incontinence type), the identification of the central part is performed (for example) by the assembly seams (different colour thread).

If the knit turns out to be too light during the suture (fixing of the arms) it is possible to fold back the arm in the suture zone.

Described below is the surgical technique implementing either of the implants (1), illustrated in FIGS. 1 and 2, aimed at treating the urethra, at the external urinary sphincter and the bladder neck, in order to restore a normal anatomical shape, after a radical prostatectomy or to suitably support an orthotopic neobladder and an external urinary sphincter, following a cystoprostatectomy.

The major surgical stages can be disclosed as follows:

After performing the prostatectomy, in a manner perfectly known by a person skilled in the art, at the time of reconstruction of the urinary contiguity (ureterovesical anastomosis), the implant (1) is positioned without tension in the surgical area.

Denonvillers' rectovesical fascia is then reconstructed in one or two layers, to create an anatomical separation barrier between the implant and the ureterovesical anastomosis to prevent risks of erosion and/or formation of fistulas. The ureterovesical anastomosis is then performed and an indwelling catheter is inserted.

The different arms of the implant (1) are then tensioned to offer suitable support to the urethra and the bladder neck. The anterior arms are fixed by means of stitches or staples to the Cooper ligament just next to the pubic symphysis, whereas the other arms are fixed to the psoas muscle/Cooper ligament (according to the size and the shape of the patient's anatomy) in a portion located between the obturator nerve and the external iliac vessels.

The balloon catheter is then inflated and the surgical procedure is completed, as standard practice.

It is to be pointed that the surgical method of the corresponding technique may be performed by open laparoscopic surgery, or robot assisted, and in the case of a radical prostatectomy or a urinary diversion with orthotopic neobladder, as indicated.

The advantages are made well apparent from the description.

1. A method for the prevention of incontinence during a prostatectomy, by means of a textile implant comprising a support portion of the urethra fitted with at least 2 arms, a method according to which:
after performing the prostatectomy, the implant is positioned, at the time of reconstruction of the urinary contiguity, without tension in the surgical area,
the rectovesical fascia is reconstructed to create an anatomical barrier/separation between the implant and the ureterovesical anastomosis,
anastomosis is performed and an indwelling catheter is inserted,
the arms are tensioned and fixed to the Cooper ligament and/or the psoas muscles,
a balloon catheter is inflated and the surgical procedure is conventionally completed.

2. The method according to claim 1, wherein when the implant has from the support portion, in a symmetrical manner, two anterior arms and two posterior arms, the anterior arms are fixed to the Cooper ligament and the posterior arms to the psoas muscle/Cooper ligament (depending on patient body shape and anatomy) in the area between the obturator nerve and the external iliac vessels.

3. The method according to claim 1, wherein when the implant has, from the support portion, in a symmetrical manner, two anterior arms and the four posterior arms, the two anterior arms are fixed to the Cooper ligament and the four posterior arms to the psoas muscle/Cooper ligament, in an area between the obturator nerve and the external iliac vessels.

4. The method according to claim 1, wherein it is implemented in the case of laparoscopic surgery.

5. The method according to claim 1, wherein it is implemented in the case of robot-assisted laparoscopic surgery.

6. An implant for the implementation of the method of claim 1, wherein it symmetrically has, from a central support portion, two posterior arms arranged in the same plane, and in alignment, and two anterior arms, in a “V” configuration, with respect to one another, and with respect to the posterior arms.

7. An implant for the implementation of the method of claim 1, wherein it symmetrically has, from a central support portion six arms arranged in the same plane and in alignment.
